(19) United States
(12) Patent Application Publication
(10) Pub. No.: US 2015/0379227 A1
(43) Pub. Date: Dec. 31, 2015

(54) MEDICAL CONDITION MANAGEMENT METHOD AND SYSTEM

(71) Applicants: Joseph Criner GERARD, Bryn Mawr, PA (US); Temple University, Philadelphia, PA (US)

(72) Inventor: Joseph Criner GERARD, Bryn Mawr, PA (US)

(21) Appl. No.: 14/766,526

(22) PCT Filed: Feb. 7, 2014
(86) PCT No.: PCT/US2014/015350

§ 371 Date: Aug. 7, 2015

Related U.S. Application Data
(60) Provisional application No. 61/761,970, filed on Feb. 7, 2013.

(51) Int. Cl. G06F 19/00; A61B 5/087; A61B 10/00; A61B 5/01

(52) U.S. Cl. CPC G06F 19/3431; A61B 5/01; A61B 5/0871; A61B 10/0051

(57) ABSTRACT

A method and system for managing a chronic medical condition, such as chronic pulmonary obstructive disorder, is disclosed. The method includes generating a baseline score for a patient; recording an exacerbation severity for a plurality of symptoms; weighting the recorded exacerbation severities; determining an exacerbation score relative to the baseline score based on at least one of the recorded or weighted exacerbation severities; assigning the exacerbation score to a category; publishing the exacerbation score and assigned category for review by a physician or medical professional; and transmitting a treatment plan to the patient, the treatment plan being prescribed by a physician and based at least in part on the exacerbation score and/or assigned category. The system includes a computer that allows the patient to perform the steps of the method and a triage center for evaluating the published exacerbation scores and prescribing and communicating a treatment plan for the patient.

### CROSS-REFERENCE TO OTHER APPLICATIONS

This application claims the benefit of U.S. Provisional Patent Application Ser. No. 61/761,970 filed Feb. 7, 2013, which is incorporated in its entirety herein by reference.

### BACKGROUND OF THE INVENTION

1. Field of the Invention

This application relates generally to a method and system for managing a chronic medical condition and, more specifically, to method and system for remotely advising a patient afflicted with chronic obstructive pulmonary disease over a communication network regarding treatment options to address an exacerbation.

2. Description of Related Art

In all classes of medical subspecialties, there are chronic illnesses that, by definition, have no “cure” and over time cause gradual or progressive deterioration to a patient's health, wellbeing, and quality of life. In addition to the continuous challenges posed by the underlying chronic disease, patients with chronic illness can occasionally experience acute exacerbations in which their symptoms temporarily worsen. These acute exacerbations can lead to emergency room visits, hospitalization, progression of the underlying disease, and even death. In many cases, acute exacerbations can be prevented (or at least their adverse impact can be diminished) when patients and their healthcare providers receive adequate advance notice regarding any changes to the patient's symptoms. A significant challenge in managing and monitoring changes to a patient's chronic condition—and subsequently identifying impending exacerbations—stems from the absence of a reference that adequately and accurately represents a patient's “normal” health. In other words, because of the effects of chronic illness, patients with chronic conditions are not normally healthy as would be defined in the general population. In addition, the “new normal” or “typical” health profile of each chronic illness patient is highly individualized, due to variations in the presentation of chronic disease as well as various co-morbidities. The absence of a “normal” or “typical” profile for comparison purposes is particularly problematic in the treatment of such patients as clinicians observing a patient's symptoms at a particular point in time have difficulty discerning whether the patient is in fact experiencing (1) a “normal” variation that is typical to the patient, (2) a degradation of that patient's overall chronic condition, or (3) an (impending or occurring) acute exacerbation.

For one of the most prevalent examples of chronic pulmonary illnesses, chronic obstructive pulmonary disease (“COPD”) exacerbations are the primary cause of hospitalization of patients with COPD. Not only do patients with COPD have a high rate of hospitalization due to COPD, about 1 in 5 patients are readmitted within 30 days of being discharged from the hospital. For example, in FY 2009, 33,477 Pennsylvanian residents were admitted for COPD exacerbation; 22.7% were readmitted within 30 days of hospital discharge. Exacerbations increase morbidity and mortality, and transiently or permanently worsen the quality of life of patients suffering from COPD. In addition, exacerbations precipitate a decline in exercise capacity and hasten the progressive loss of lung function. Exacerbations consume the majority of COPD costs, with expenditures related to exacerbations reaching $49.9 in 2010. Because such exacerbations can be frightening to the patient, it is common for patients experiencing an exacerbation to immediately seek medical attention at an emergency room or other healthcare provider. However, not all exacerbations are severe enough to warrant a visit to an emergency room or a personal visit to a physician. Exacerbations resulting in unnecessary visits to an emergency room or physician can be costly to the patient and the insurance providers, requiring them to pay for the costs of treating mild exacerbations that, although unpleasant, are not uncommon for the patient's then-current medical condition and did not warrant in-person medical attention.

In light of the above issues, a method and system for managing chronic illnesses is desired that can establish a patient specific baseline health profile, remotely determine the severity of the patient's exacerbations, and remotely propose a treatment for the patient.

### BRIEF SUMMARY OF THE INVENTION

A simplified summary is provided herein to help enable a basic or general understanding of various aspects of exemplary, non-limiting embodiments that follow in the more detailed description and the accompanying drawings. This summary is not intended, however, as an extensive or exhaustive overview. Instead, the sole purpose of the summary is to present some concepts related to some exemplary non-limiting embodiments in a simplified form as a prelude to the more detailed description of the various embodiments that follow.

In one aspect, the method and system described herein relate to determining a reference baseline for use in assessing the condition of patients with a chronic illness. The baseline acts as a measurement of the severity of a chronic patient's “normal” symptoms, against which an exacerbation can be compared.

In another aspect, the method and system described herein relate to determining a patient's exacerbation score. That is, a patient may answer a series of questions regarding severity of symptoms related to their chronic illness. These answers are compared to the baseline score. The patient's answers and comparison to the baseline score can then be used to determine an exacerbation score. These scores may be determined during periods of exacerbation, on a periodic basis, or the like. Severity of symptoms may also be recorded directly by sensors, manual input, or the like.

In a further aspect, the method and system described herein relate to the treatment of a patient based in part on their particular exacerbation score. The exacerbation score may be reported to a central location, for example, a triage center, thereby preventing a costly physical visit to a physician or clinician's office by the patient unless necessary.

According to one example, the disclosure herein describes a method for managing a chronic medical condition, comprising the steps of generating a baseline symptom severity score for a patient; recording, by the patient, an exacerbation severity for each of a plurality of symptoms related to the medical condition; weighting at least one of the recorded exacerbation severities according to a predetermined weighting factor corresponding to the symptom associated with the recorded exacerbation severity; determining an exacerbation score relative to the baseline score based at least in part on at least one of the recorded or weighted exacerbation severities; assigning the exacerbation score to a category; publishing the exacerbation score and assigned category for review by a physician or medical professional; and transmitting a treatment plan to the patient, the treatment plan being prescribed by a physician and based at least in part on the exacerbation score and/or assigned category.

According to other examples of the above method, the predetermined weighting factors are customized by a physician; the step of adjusting the baseline symptom severity score at least in part on a plurality of determined exacerbation scores; further comprises the step of performing triage based on the exacerbation score and/or assigned category; at least one sensor is used to determine an exacerbation severity; the medical condition is chronic obstructive pulmonary disorder; the above steps are performed periodically; and the plurality of symptoms related to the medical condition include at least one from the group consisting of: breathlessness, sputum quantity, sputum color, sputum consistency, peak expiration flow, and temperature.

According to another example, the disclosure herein describes a system for managing a chronic medical condition, comprising: a computer configured to: receive input from a patient related to an exacerbation severity for each of a plurality of symptoms related to the medical condition; weight at least one of the exacerbation severities according to a predetermined weighting factor corresponding to the symptom associated with the exacerbation severity; determine an exacerbation score relative to a baseline symptom severity score based at least in part on at least one of the inputted or weighted exacerbation severities; assign the exacerbation score to a category; publish the exacerbation score and/or the assigned category related to the patient; and receive and report a treatment plan to the patient; and a triage center for evaluating the published exacerbation scores, wherein the triage center prescribes the treatment plan for the patient based at least in part on the published exacerbation score and/or assigned category and communicates the treatment plan to the computer for review by the patient.

According to other examples of the above system, the predetermined weighting factors are customized by a physician or clinician; The system of claim 9, wherein the baseline symptom severity score is adjusted at least in part on a plurality of determined exacerbation scores; at least one sensor is operatively connected to the computer and used to determine an exacerbation severity; the medical condition is chronic obstructive pulmonary disorder; the computer receives input from a patient periodically; and the plurality of symptoms related to the medical condition include at least one from the group consisting of: breathlessness, sputum quantity, sputum color, sputum consistency, peak expiration flow, and temperature.

These and other embodiments are described in more detail below.

### BRIEF DESCRIPTION OF SEVERAL VIEWS OF THE DRAWING

The invention may take physical form in certain parts and arrangement of parts, embodiments of which will be described in detail in this specification and illustrated in the accompanying drawings which form a part hereof and wherein:

FIG. 1 illustrates a flow diagram of one embodiment of the method of the present disclosure;

FIG. 2 illustrates a login screen in one embodiment of an app of the present disclosure;

FIG. 3 illustrates a home screen in one embodiment of an app of the present disclosure;

FIG. 4 illustrates a list of completed reports that may be shown in one embodiment of an app of the present disclosure;

FIG. 5 illustrates details of a selected report that may be shown in one embodiment of an app of the present disclosure;

FIG. 6 illustrates a screen for reporting breathlessness in one embodiment of an app of the present disclosure;

FIG. 7 illustrates a screen for reporting sputum quantity in one embodiment of an app of the present disclosure;

FIG. 8 illustrates a screen for reporting sputum color in one embodiment of an app of the present disclosure;

FIG. 9 illustrates a screen for reporting sputum consistency in one embodiment of an app of the present disclosure;

FIG. 10 illustrates a screen for reporting peak flow measurements in one embodiment of an app of the present disclosure;

FIG. 11 illustrates a screen for reporting temperature in one embodiment of an app of the present disclosure;

FIG. 12 illustrates a screen for reporting minor symptoms in one embodiment of an app of the present disclosure;

FIG. 13 illustrates a summary screen of a report in one embodiment of an app of the present disclosure;

FIG. 14 illustrates a summary confirmation request for a newly completed report in one embodiment of an app of the present disclosure;

FIG. 15 illustrates a confirmation screen for a newly completed report in one embodiment of an app of the present disclosure;

FIG. 16 illustrates a sample flow chart for treatment intervention that may be prescribed to a user with mild exacerbation;

FIG. 17 illustrates a sample flow chart for treatment intervention that may be prescribed to a user with moderate exacerbation; and

FIG. 18 illustrates a sample flow chart for treatment intervention that may be prescribed to a user with extreme exacerbation.

### DETAILED DESCRIPTION OF THE INVENTION

Certain terminology is used herein for convenience only and is not to be taken as a limitation on the present invention. Relative language used herein is best understood with reference to the drawings, in which like numerals are used to identify like or similar items. Further, in the drawings, certain features may be shown in somewhat schematic form.

It is also to be noted that the phrase “at least one of”, if used herein, followed by a plurality of members herein means one of the members, or a combination of more than one of the members. For example, the phrase “at least one of a first widget and a second widget” means in the present application: the first widget, the second widget, or the first widget and the second widget. Likewise, “at least one of a first widget, a second widget and a third widget” means in the present application: the first widget, the second widget, the third widget, the first widget and the second widget, the first widget and the third widget, the second widget and the third widget, or the first widget and the second widget and the third widget.

The method and system described herein involves the timely treatment of chronic illness exacerbations. In one aspect, such treatment uses telemedicine combined with a management program. Telemedicine combined with a management program can decrease the frequency and severity of exacerbations that would otherwise occur without the combination of a telemedicine and management program, and improve daily symptom control, lung function, dyspnea, and improved peak flow and daily activity status. Telemedicine combined with a management program works by facilitating early recognition and timely treatment of impending acute exacerbations, as well as by monitoring the duration and severity of acute exacerbations. For example, incorporating daily telemedicine-based symptom reporting through a management program could decrease hospitalizations, mortality, and reduce the frequency and severity of acute exacerbations in high risk patients.

It should be noted that for simplicity, chronic obstructive pulmonary disorder (COPD) is used throughout as an example chronic illness for use with the method and system described herein. However, any chronic illness, including other chronic pulmonary illnesses, is envisioned to be within the scope of the present disclosure, and the use of COPD is not intended to be a limiting use.

Additionally, it should be noted that for simplicity a “diary” application on a patient's computer or mobile device is used herein as a telemedicine management program. However, any management mechanism generally used by the patient remotely with respect to a physician or clinician is envisioned within the scope of the present disclosure. For example, the patient may record daily symptoms in a record book at home and report them to a doctor by phone or mail. Therefore, a diary application should not be viewed as a limiting embodiment.

One aspect of the present method and system described herein relates to determining a reference baseline for use in assessing the condition of patients with a chronic illness. The baseline, as used herein, is a measurement of the severity of a chronic patient's “normal” symptoms, against which an exacerbation can be compared. That is, the definition of exacerbation as used herein is a worsening in a patient's symptoms compared to their baseline, not the traditional definition used in many COPD interventional trials denoted by the use of antibiotics, steroids or both.

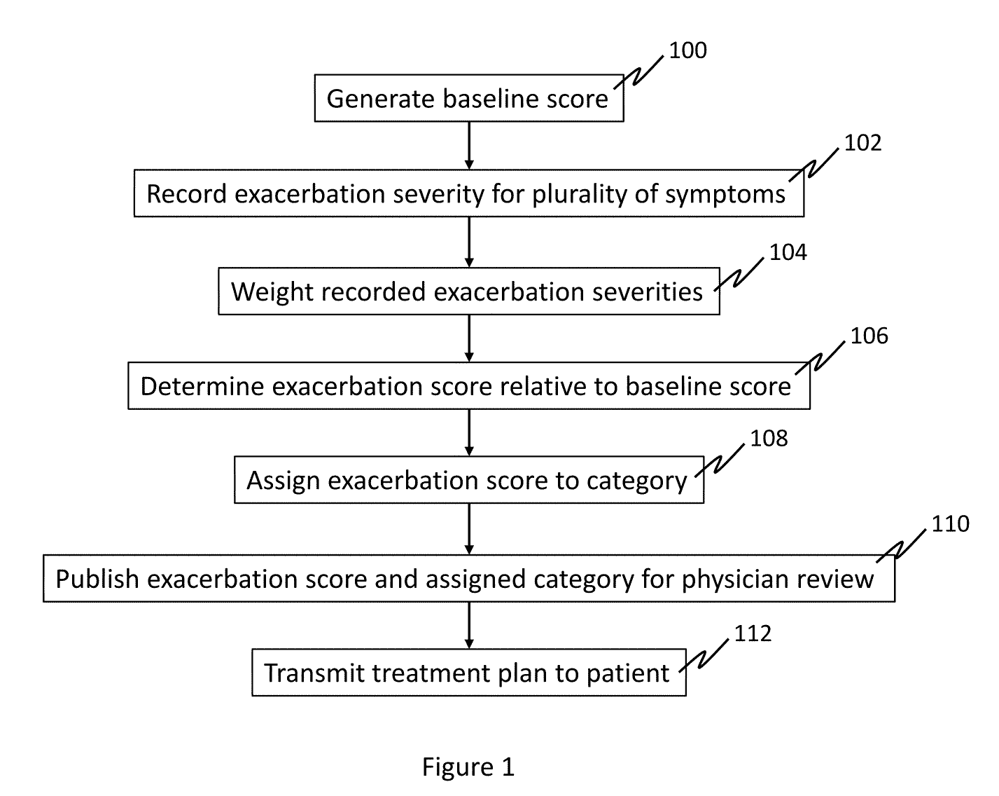

Turning now to the figures, FIG. 1 illustrates a flow diagram of one embodiment of the method of the present disclosure. A first step in the method is to generate a baseline score 100 for each patient. Although the severity of symptoms for a chronic patient are likely to be worse than that of the general population or patients without the chronic illness, the determination of acute exacerbations of the chronic patient's illness are best understood with respect to each chronic patient's “normal” conditions. Therefore, the determination of a baseline “normal” and any particular exacerbations are generally patient specific. Once the reference baseline has been established, it may be re-assessed after a period time to reflect changes in (i.e., update) the “normal” state of the patient's condition. In still one or more embodiments, the baseline may be automatically established or re-assessed, or manually established and re-assessed by a physician or clinician.

After a baseline score has been generated, a patient may then begin recording exacerbation severities for a plurality of symptoms 102, for example, on a regular basis or during periods of acute exacerbation. Once a patient's symptom severities have been recorded 102, the scores are weighted according to a predetermined algorithm 104. The effect of weighting scores is to give particular symptoms, for example, those that may be better or worse indicators of an acute exacerbation, an appropriate factor when determining the severity of the patient's current health state. As described in more detail below, these weighting factors may be customizable. Next, an exacerbation score is determined 106 using the weighted symptom severities and according to a predetermined algorithm. In many embodiments, this algorithm determines a score relative to the patient's baseline score. Again, the algorithm is described in more detail below and may be customizable. Next, the score may be assigned to a category 108 in order to classify the severity of the patient's exacerbation. The patient's score and exacerbation category can then be published to a physician 110, located remotely, for example, for further review and determination of a treatment plan. The treatment plan may then be transmitted back to the patient 112.

In one or more embodiments, the above method is performed using a diary application (hereinafter “the app”), representing a telemedicine management application, accessible by the patient. A new patient may enroll in the app by establishing user data (e.g., username, password, patient history, demographic information, etc.) corresponding to that patient and downloading the app onto their computer or mobile device. The app serves to simplify the patient's recording of the severity of their symptoms and subsequent transmission of this data to a physician or clinician. Each set of data entries regarding symptom severity is herein referred to as a “check-in”. Once downloaded, the patient may then, for example, use their email/username and password, or the like, to login. As previously discussed, the app is used herein as an example of a telemedicine management application, and should not be seen as a limiting embodiment. Other telemedicine management programs may include website (server-side) applications, hand written diaries, and the like.

In one or more embodiments, a baseline is established during the first 14 days in an enrollment/baseline establishment phase. In other embodiments, this phase may be less than or greater than 14 days. In other embodiments, a baseline may be established after a set amount of data is recorded, rather than a set period of time. In still other embodiments, the baseline may be established prior to or during the patient's enrollment based on previously recorded and/or observed data.

During the enrollment phase, the app operates as it normally would by accepting data entry from the patient regarding their symptom severities; however, no exacerbation determinations are ultimately made. Rather, each time the patient checks-in, the data is recorded for establishing a baseline. In one or more embodiments, patients may complete any number of check-ins during the establish baseline phase, but only the first check-in from each day may be used to calculate the patient's baseline. Each check-in can appear in the patient's check-in history. A check-in preview and detail can further include the number of days into the establish baseline phase (and/or days remaining)—e.g. Day 3 of 14 of establish baseline phase or 11 Days of establish baseline remaining—with a progress bar (e.g. 14 bars with an additional bar shaded each day). Once the system obtains a first check-in on the final day of the establish baseline phase, the app can determine the patient's baseline. The app then shifts to a check-in and algorithm phases according to another aspect of the method and system described herein.

This check-in and algorithm related aspect is directed to determining a patient's exacerbation score. In or more embodiments, the exacerbation score is determined on a periodic basis, for example, daily. In one or more embodiments, the exacerbation score determination is based in part on data submitted by the patient and/or recorded from the patient (i.e., from sensors). Examples of sensors that may be used within the present disclosure include, but are not limited to, spirometers, thermometers, pulse oximeters, or the like.

During the check-in phase, the patient may log in to the app and record the severity of symptoms associated with their chronic illness. In one or more embodiments, patients may log in periodically, for example, daily. In other embodiments, patients may log in only when they believe their symptoms to be exacerbated or are suffering acute attacks. During the check-in procedure, patients answer questions regarding the severity of various symptoms they may be experiencing. In some embodiments, certain symptoms may require that a sensor be used to measure and/or quantify a symptom. Depending on the symptom, more than one sensor reading may be required for a particular symptom. In these cases, the app may record all of the readings, an average of all readings, only the most extreme readings, or some other variation known to those skilled in the art. In still other embodiments, the app may likewise use one or more of the readings in determining a patient's exacerbation score, independent of the number of actual recordings.

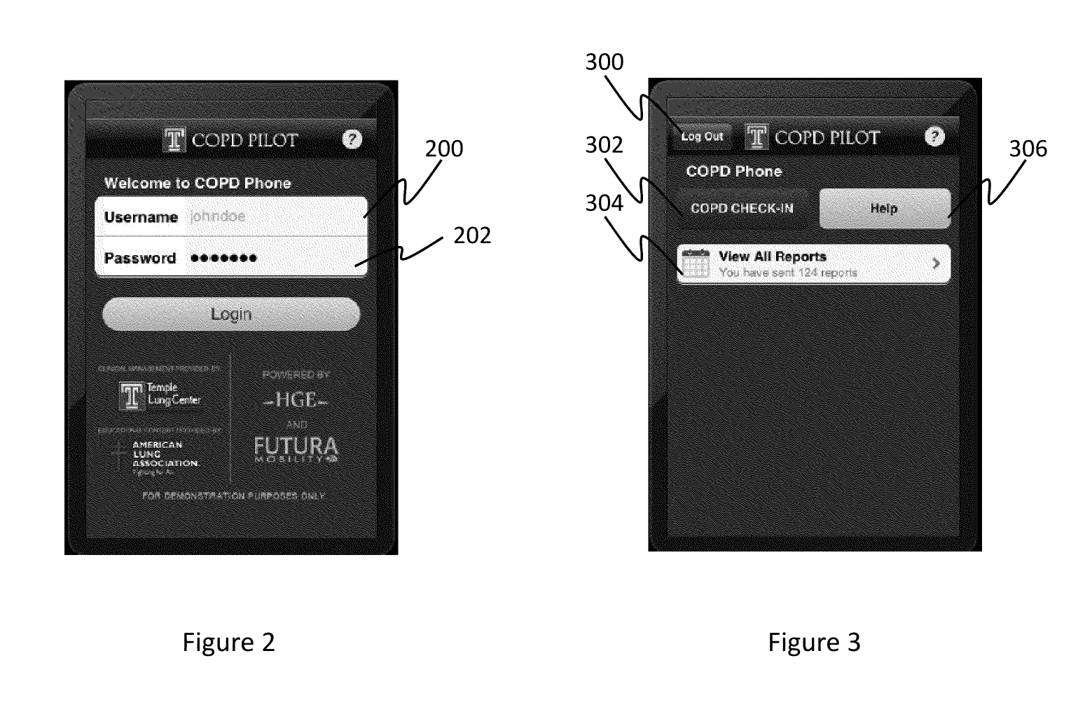

FIGS. 2-13 illustrate the check-in process and various symptom related questions that may be asked in one embodiment of the app. For example, FIG. 2 illustrates a login page first seen by a user after launching the app. At the login page, the user may enter their login information, for example a username 200 and password 202 to login. Although not shown, this screen may also be used to provide telephone numbers or similar contact information for help regarding the app and/or current medical questions.

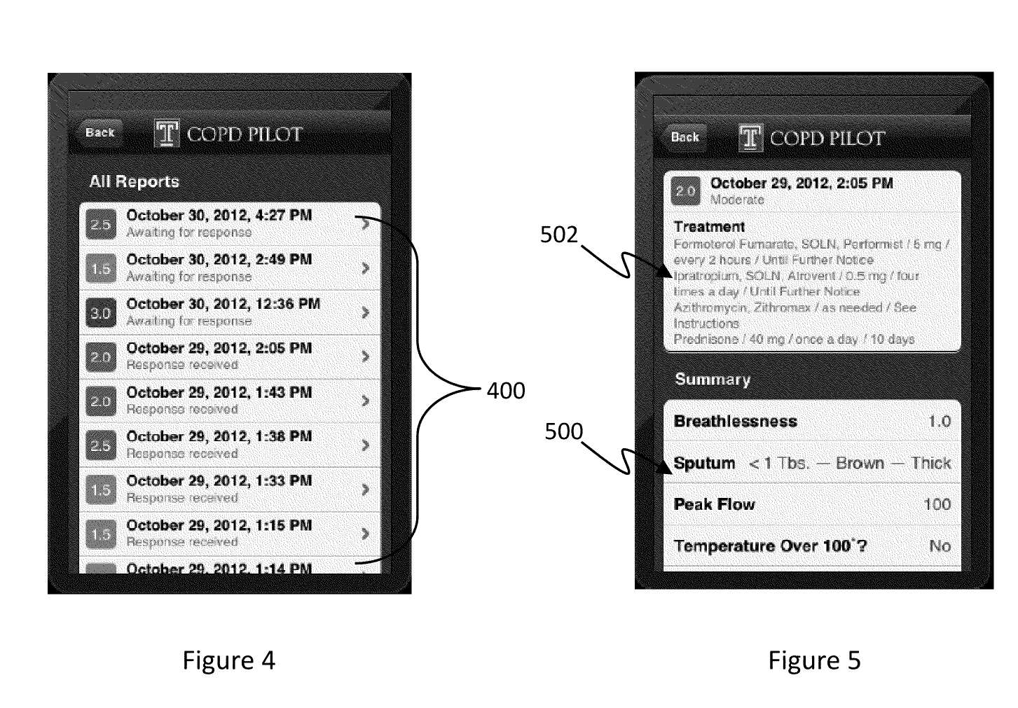

FIG. 3 illustrates the home page a user may see immediately after logging in. From this screen, the user can have a variety of options to select “Log Out” 300, “Check-In” 302, “View All Reports” 304, or seek “Help” 306. Selecting “Log Out” 300 logs the user out of the app. In some embodiments, the user may be prompted to confirm that they would like to log-out. In some embodiments, logging out may also remove any data from the device where the app is located as an added security feature. Selecting “View All Reports” 304 may provide the user with a list of all reports they have submitted, as shown in FIGS. 4 and 5. “Help” 306 may take the user to a help screen (not shown). In some embodiments, the help screen may provide contact information for the app and/or medical questions. In other embodiments, selecting “Help” may provide users with information describing the action of each button on the screen, frequently asked questions regarding how to use the app, or the like. In other embodiments not shown, the home screen may also contain buttons to show various settings, initiate an email with a physician, clinician, or nurse wherein the app itself functions as a mail client, or provide various hyperlinks to educational resources and available clinical trials.

As shown in FIG. 4, a list 400 of all completed reports may be shown to the user if requested. This list 400 may include the dates and times of submitted reports, as well as the determined exacerbation score and the current status of the report. From this list, the user may select a report to view more details. As shown in FIG. 5, the details related to the selected report additionally may include a summary 500 of the symptom severities reported, as well as the proposed treatment plan 502. Other details related to the report may be shown in other embodiments without diverging from the scope of this disclosure.

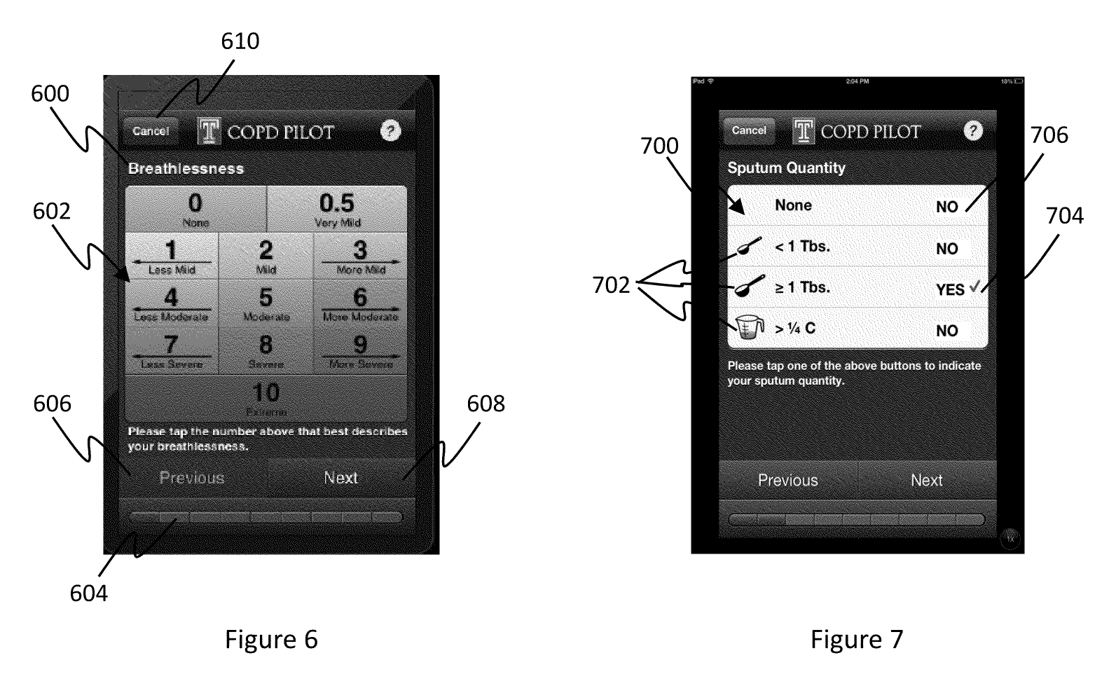

When the user selects “Check-In” 302, they may begin reporting symptom severities in a series of screens related to each symptom analyzed as part of the method and system. In the example described herein, the user is first taken to a screen for reporting their breathlessness, as shown in FIG. 6. The symptom 600 corresponding to each screen may be labeled on the screen. In this example, breathlessness may be rated from 0-10 (0 being none; 10 being extreme), by selecting corresponding number on the screen, wherein the numbers are shown in a manner similar to a keypad 602. In addition to rating breathlessness with numerical values, colors correspond to each value to provide a visual trigger as to the range (green to yellow to orange to red). It should be noted that other embodiments may provide a greater range of available numerical values, or more options within a range. Other embodiments may also use a different color scheme, or no color scheme. In still other embodiments, the ratings may be shown as a scale, and selected by a sliding bar, or other known methods for selecting numbers as known to those of ordinary skill in the art.

It should also be noted that through the check-in process, a progress bar 604 may be shown to illustrate the user's progress. Although located along the bottom edge of the screen in the present example, the progress bar could be located anywhere on the screen or be of any form. For example, other forms of progress indicators could show the number of screens remaining, the number of screens completed with respect to the total number of screens, a percentage of the total symptoms reported, or the like. Upon selecting a proper response to the indicated symptom, the app may automatically advance to the next screen. Other embodiments, however, may highlight, or otherwise indicated, the selected response and wait for the user to select a “Next” option 606. Likewise, the user may select “Previous” 608 to return to the previous screen for reviewing or modifying a response (if available). A user may also cancel 610 the check-in process at any time.

Next, the user is asked to indicate their sputum quantity for the past 24 hours, as shown in FIG. 7. The user is provided with 4 choices 700: (1) none; (2)<1 Tbs.; (3)≧1 Tbs.; and (4)>¼ cup. Of course, fewer or greater choices may be provided depending on the embodiment, or the time frame may be shorter or greater than 24 hours. An image 702 related to the quantity may also be shown next to the quantity so that the user has a better understanding of each option. For example, as shown in FIG. 7, a measuring cup is shown next to the >¼ cup option, while a small spoon is shown next to the <1 Tbs. option. A checkmark and “Yes” 704 or “No” 706 may indicate which option has been selected.

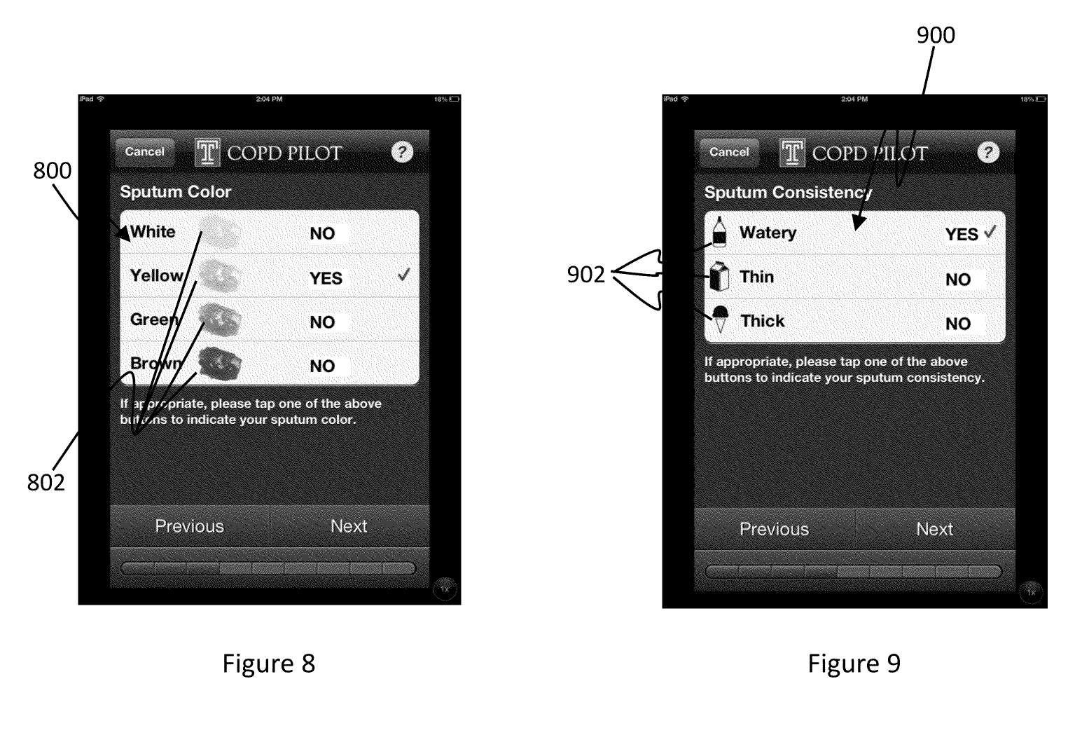

As shown in FIGS. 8 and 9, other screens may ask the user to rate their sputum color 800 and/or sputum consistency 900 over the past 24 hours. For example, the sputum color may be white, yellow, green, or brown. In some embodiments, the most severe color should be selected, whereas in other embodiments all colors brought up by the patient may be selected. Color swatch images 802 may be shown next to each option to help the user better identify the appropriate response by way of comparison. Sputum consistency may be watery (e.g., water), thin (e.g., milk), or thick (e.g., ice cream). The consistency is a subjective measurement that can be generalized by the user based on the consistency of their sputum over the past 24 hours. Again, images or icons 902 may be used to help the user make comparisons. Fewer or greater options and shorter or greater time frames may be provided with respect to these symptoms as well.

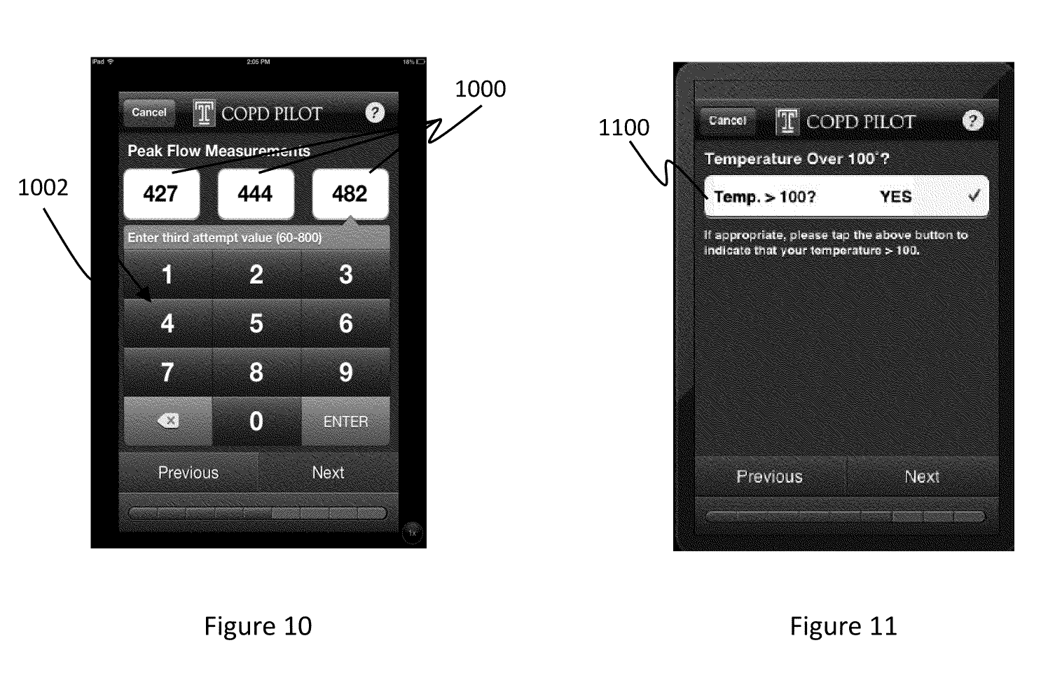

A screen corresponding to peak flow measurements is shown in FIG. 10. A peak flow meter can be used to measure the user's peak expiratory flow (PEF). In the present example, the user is asked to complete three measurements 1000. If the measurements have a variance of greater than, for example, 20%, the user may be warned that the entries may not be acceptable. In some embodiments, a peak flow meter may be directly connected to the app and/or device used by the user, thereby directly imputing each PEF measurement into the app. In other embodiments, the user may be tasked with manually entering the recordings using, for example, a keypad 1002.

Next, as shown in FIG. 11 the user is asked whether their temperature is greater than 100° F. Similar to the PEF measurement, a thermometer may be used to directly record the user's temperature and select that the temperature is greater than 100° 1100 if that is the case. In other embodiments, the user may manually have to make such a selection. In still other embodiments, the user may be asked if their temperature exceeded 100° at any point in a predefined period (e.g., 24 hours).

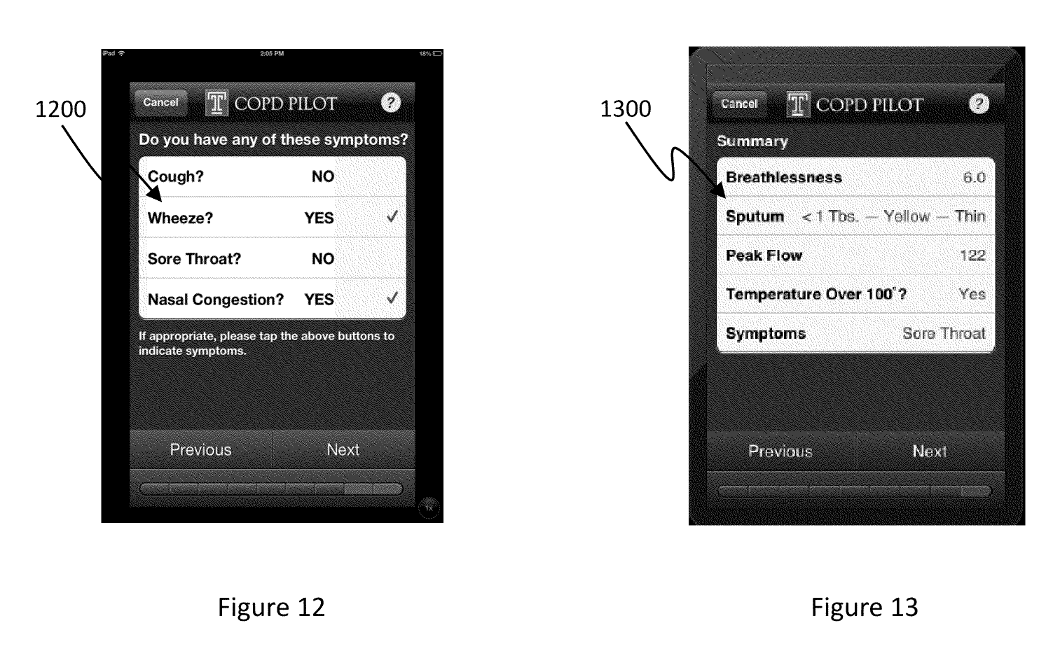

FIG. 12 illustrates a screen related to minor symptoms 1200, including: coughing, wheezing, sore throat, and nasal congestion. The user can select any or all minor symptoms that they have experienced over the past 24 hours. Of course, other embodiments may include greater or fewer options, or a shorter or longer time frame.

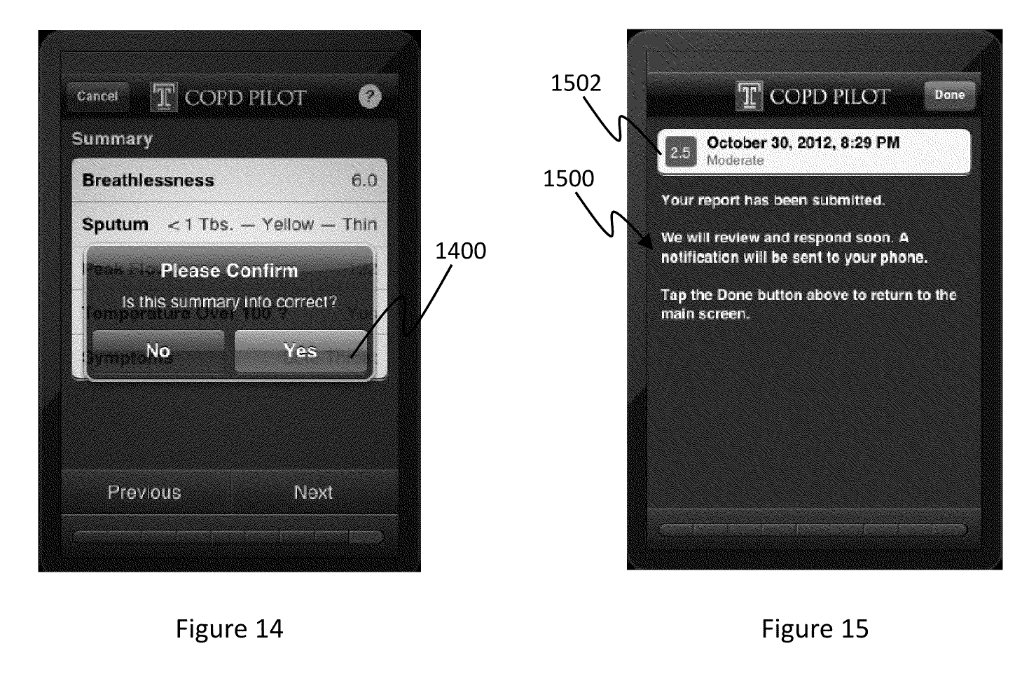

After indicating which minor symptoms were experienced, the user may be taken to a summary screen, as in FIG. 13. The summary screen provides a review of each of the symptoms and severities indicated by the user 1300. The user may then confirm this report 1400, as shown in FIG. 14, or return to a particular symptom screen to correct an entry. Once confirmed, an exacerbation score can be tabulated and the report can be saved and submitted for review. A confirmation screen, as shown in FIG. 15, confirms these actions 1500 and may report a determined exacerbation score and category 1502—the determination of which is discussed below.

It is to be noted that the above example of symptoms to be reported are not intended to be limiting, rather they are but a single example of symptoms that could be used with respect to COPD. Furthermore, in addition to individual symptoms, the app may ask a user questions relating to their overall health and/or quality of life. For example, the app could seek to establish the patient's dyspnea (modified Borg Score) and/or the Duke Activity Status Index (DASI). It should also be noted that the check-in process is envisioned to be highly customizable. For example, the check-in process could be customized between physicians or clinicians. The check-in process may also be customizable between chronic illnesses.

After a patient has answered all questions related to the severity of their symptoms, and the check-in is successfully completed, the app shifts to an algorithm phase wherein an algorithm is applied to the check-in data. That is, the applied algorithm determines an exacerbation score for the patient's symptoms by comparing the data values for that check-in to the patient's baseline score and adding points (starting with zero) based on changes from the baseline values. It is important to note that any algorithm may be used to calculate an exacerbation score. For example, in some embodiments, symptoms may be given more weight than the same symptoms in other embodiments. Each physician or clinician may weight symptoms as they see fit. In some embodiments, weighting is performed by associating different score values with a given symptom if the severity of that symptom increases above a certain threshold. In other embodiments, weighting may be performed by altering the threshold(s) according which points are assigned. In still other embodiments, a physician or clinician may wish to alter both the score and threshold values. Various embodiments may also perform any mathematical operation on the scores associated with each symptom. Still other algorithms that assign scores based on reported symptom severities to determine an overall exacerbation score are envisioned to be within the scope of the present disclosure.

For purposes of this disclosure, the following example of an algorithm is described herein. In the following example, a positive score value is associated with each symptom if the severity of the symptom matches a particular condition. The sum of all points per the calculation below is the exacerbation score. In one example, scores for each symptom are tabulated as follows:

| | Score | Condition |
|---|---|---|
| Major Symptoms | | |
| Breathlessness | +1.0 | if ≧3 points above baseline |
| Sputum Quantity | +0.5 | if change to greater amount from baseline |
| Sputum Color | +0.5 | if change in color from baseline |
| Sputum Consistency | +0.5 | if change from baseline and change is from |
| | | none, watery, or thin to thick |
| Peak Flow | +1.0 | if ≦80% of baseline |
| Temperature | +0.5 | if over 100° F. |
| Minor Symptoms | | |
| Cough | +0.5 | if ≧2 minor symptoms |
| Wheeze | +0.5 | if ≧2 minor symptoms |
| Sore Throat | +0.5 | if ≧2 minor symptoms |
| Nasal Congestion | +0.5 | if ≧2 minor symptoms |

After the above scores are summed to determine an exacerbation score, the exacerbation score may then be categorized as falling into one of four severity categories: None (0-0.5); Mild (1.0-1.5); Moderate (2.0-2.5); and Severe (≧3.0). Interventions and treatments may be determined based upon the severity category of the exacerbation score for the patient's current check-in. A score equal to or greater than 1 is considered an exacerbation requiring an intervention. The exacerbation scores may also be color coded according to their severity.

Still another aspect of the present method and system described herein relates to the treatment of a patient based in part on their particular exacerbation score. In one or more embodiments, treatment methods may be automated or determined by a physician. In some embodiments, a triage center may facilitate determining treatment plans for a plurality of patient's based on their exacerbation scores.

If the algorithm phase determines that the check-in does not require an intervention, the app can return to the check-in phase for that user. However, if the algorithm phase determines that the check-in does require an intervention, the app can flag the check-in and move to the intervention phase for that user.

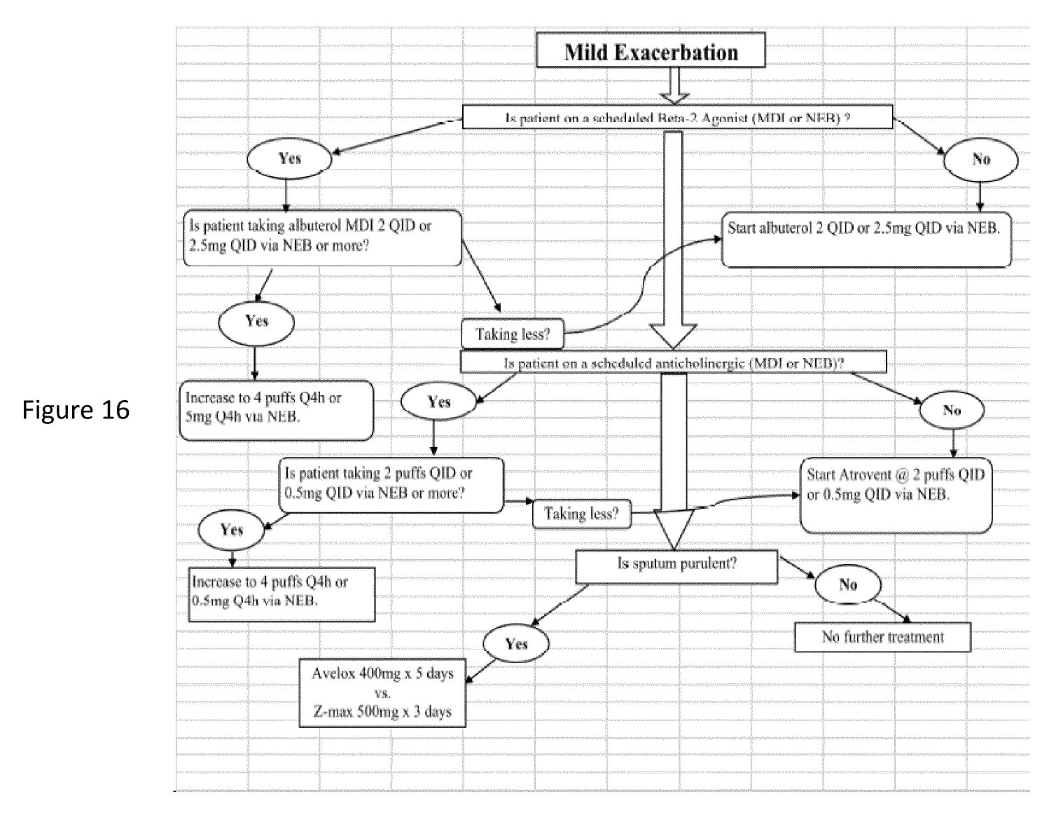

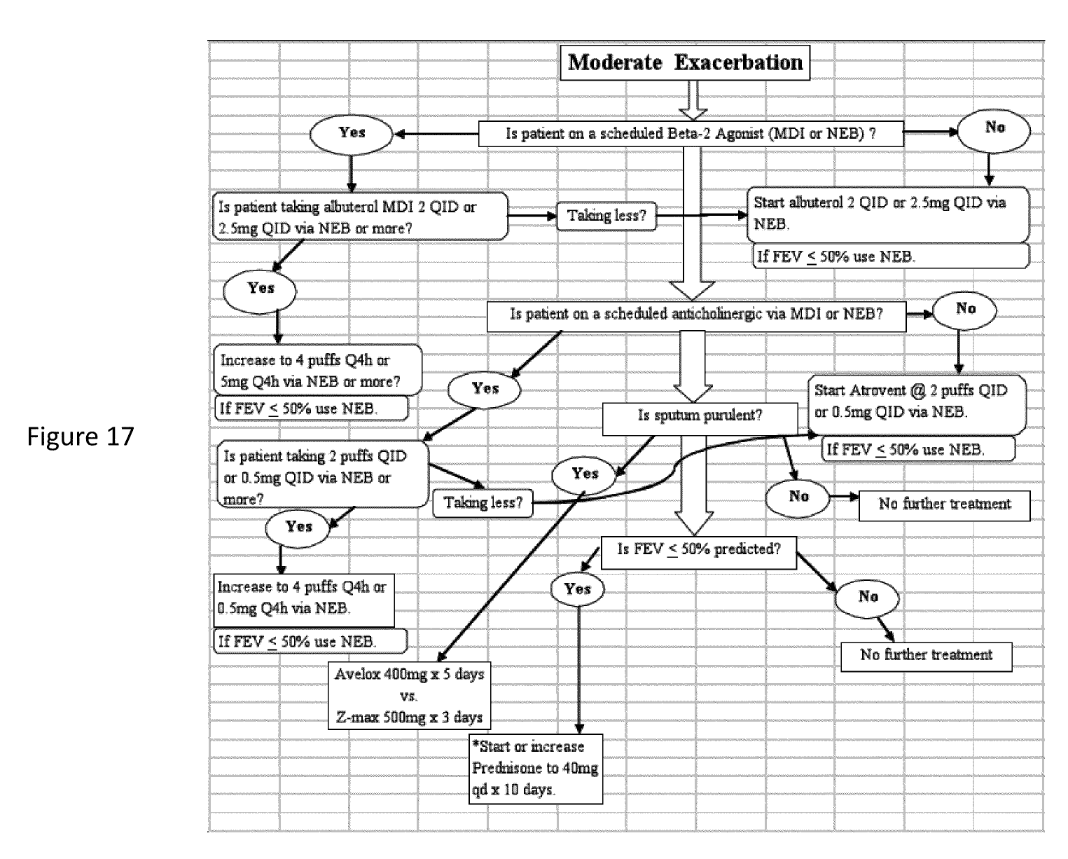

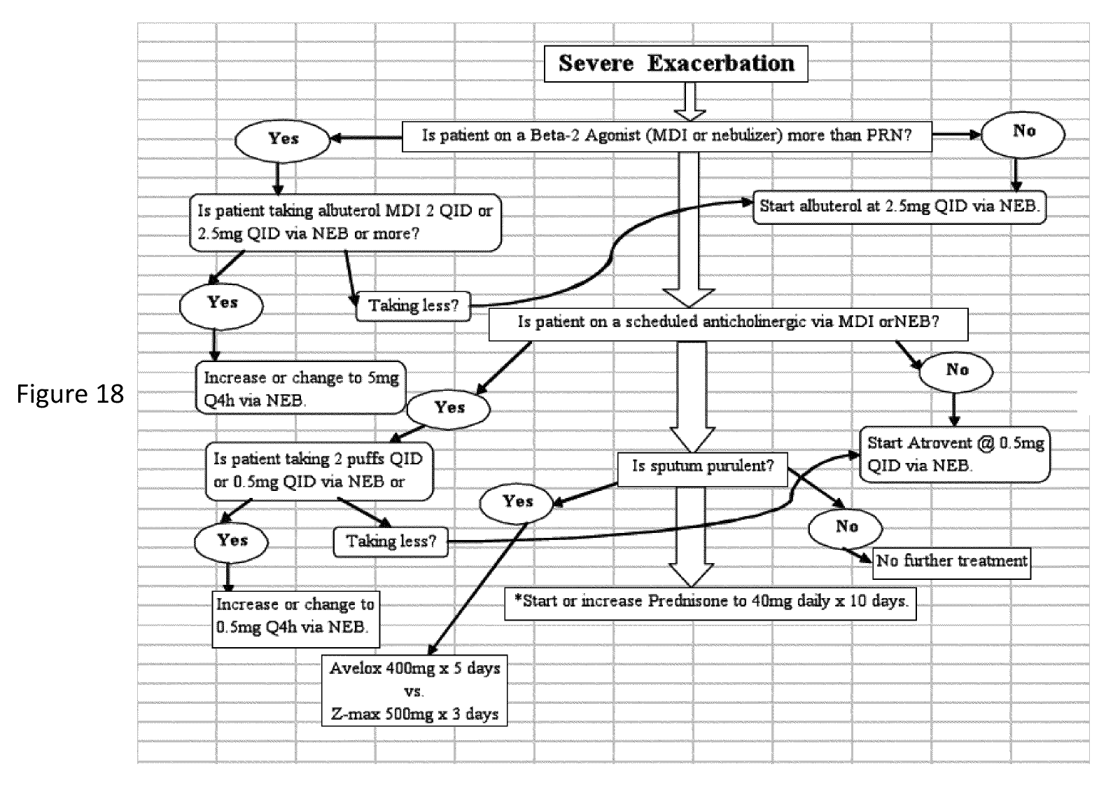

FIGS. 16-18 illustrate sample flow charts for treatment intervention that may be prescribed to the user based on their symptom severity report and/or exacerbation score. FIG. 16 illustrates treatment plans for a mild exacerbation; FIG. 17 illustrates treatment plans for a moderate exacerbation; and FIG. 18 illustrates treatment plans for a severe exacerbation. These treatment plans may further be based on the patient's history and current treatment for their chronic illness.

It should be noted that while the treatment plans may be automated, in many embodiments, it may be desirable for a physician or clinician to review the symptom severity reports and/or exacerbation score, along with the patient's history and current treatment, and design a custom treatment for that patient. In some embodiments, this may be performed via a central location. That is, the user may submit their report automatically through the app (or manually via phone, external computer, website, or the like) to a triage center. The triage center may then manage the incoming reports to determine which patients have the greatest need. For example, patients with reported severe exacerbations may be tended to first. The triage center may then forward the reports to physicians or clinicians working within the triage center or remotely for immediate review and treatment prescription.

Once a treatment plan has been prescribed, it may be presented to the user. In some embodiments, the plan may be “pushed” to the user via a notification by the app. In some embodiments, the patient may receive a phone call alerting them of a plan. Such phone calls could also be made to the patient following prescribing a treatment plan, for example, 24 and 96 hours after the intervention. In severe cases, an ambulance or similar emergency response may be initiated by the triage center on behalf of the patient.

What is claimed is:

1. A method for managing a chronic medical condition, comprising the steps of:
a. generating a baseline symptom severity score for a patient;
b. recording, by the patient, an exacerbation severity for each of a plurality of symptoms related to the medical condition;
c. weighting at least one of the recorded exacerbation severities according to a predetermined weighting factor corresponding to the symptom associated with the recorded exacerbation severity;
d. determining an exacerbation score relative to the baseline score based at least in part on at least one of the recorded or weighted exacerbation severities;
e. assigning the exacerbation score to a category;
f. publishing the exacerbation score and assigned category for review by a physician or medical professional; and
g. transmitting a treatment plan to the patient, the treatment plan being prescribed by a physician and based at least in part on the exacerbation score and/or assigned category.

2. The method of claim 1, wherein the predetermined weighting factors are customized by a physician.

3. The method of claim 1, further comprising the step of adjusting the baseline symptom severity score at least in part on a plurality of determined exacerbation scores.

4. The method of claim 1, further comprising the step of performing triage based on the exacerbation score and/or assigned category.

5. The method of claim 1, wherein at least one sensor is used to determine an exacerbation severity.

6. The method of claim 1, wherein the medical condition is chronic obstructive pulmonary disorder.

7. The method of claim 1, wherein steps b-g are performed periodically.

8. The method of claim 1, wherein the plurality of symptoms related to the medical condition include at least one from the group consisting of: breathlessness, sputum quantity, sputum color, sputum consistency, peak expiration flow, and temperature.

9. A system for managing a chronic medical condition, comprising:
a computer configured to:
receive input from a patient related to an exacerbation severity for each of a plurality of symptoms related to the medical condition;
weight at least one of the exacerbation severities according to a predetermined weighting factor corresponding to the symptom associated with the exacerbation severity;
determine an exacerbation score relative to a baseline symptom severity score based at least in part on at least one of the inputted or weighted exacerbation severities;
assign the exacerbation score to a category;
publish the exacerbation score and/or the assigned category related to the patient; and
receive and report a treatment plan to the patient; and
a triage center for evaluating the published exacerbation scores,
wherein the triage center prescribes the treatment plan for the patient based at least in part on the published exacerbation score and/or assigned category and communicates the treatment plan to the computer for review by the patient.

10. The system of claim 9, wherein the predetermined weighting factors are customized by a physician or clinician.

11. The system of claim 9, wherein the baseline symptom severity score is adjusted at least in part on a plurality of determined exacerbation scores.

12. The system of claim 9, wherein at least one sensor is operatively connected to the computer and used to determine an exacerbation severity.

13. The system of claim 9, wherein the medical condition is chronic obstructive pulmonary disorder.

14. The system of claim 9, wherein the computer receives input from a patient periodically.

15. The system of claim 9, wherein the plurality of symptoms related to the medical condition include at least one from the group consisting of: breathlessness, sputum quantity, sputum color, sputum consistency, peak expiration flow, and temperature.
